(19) United States
(12) Patent Application Publication
(10) Pub. No.: US 2020/0405596 A1
(43) Pub. Date: Dec. 31, 2020

(54) COMPOUND, COMPOSITION AND METHOD FOR PROTECTING SKIN FROM TEMPERATURE EXTREMES

(71) Applicant: Entry Point International, LLC, Wilmington, MA (US)

(72) Inventor: Peter Martin Dillon, Topsfield, MA (US)

(73) Assignee: Entry Point International, LLC, Wilmington, MA (US)

(21) Appl. No.: 16/835,514

(22) Filed: Mar. 31, 2020

Related U.S. Application Data
(60) Provisional application No. 62/867,522, filed on Jun. 27, 2019.

(51) Int. Cl. A61K 8/25; A61K 8/81; A61Q 17/00

(52) U.S. Cl. CPC A61K 8/25; A61Q 17/00; A61K 8/8129

(57) ABSTRACT

The present invention is directed toward a skin protection composition and method of use, for skin protection against extreme temperatures. The present disclosure teaches a silica-based composition applied to skin prior to extreme temperature exposure to protect skin from said exposure. The composition is formulated into a variety of topically-applied compositions and means for dispensing said compositions.

### CROSS REFERENCE TO RELATED APPLICATIONS

This application claims the benefit of U.S. Provisional Patent Application 62/867,522 entitled “COMPOUND, COMPOSITION AND METHOD FOR PROTECTING SKIN FROM TEMPERATURE EXTREMES”, filed on Jun. 27, 2019, the entire contents of which are incorporated herein by reference in its entirety for all purposes.

### FIELD OF THE INVENTION

The present invention is directed toward a skin protection composition and method of use, for skin protection against extreme temperatures. The present disclosure teaches a silica-based composition applied to skin prior to extreme temperature exposure to protect skin from said exposure. The composition is formulated into a variety of topically-applied compositions and means for dispensing said compositions.

### BACKGROUND OF THE INVENTION

Skin is the human body's largest organ and is part of the integumentary system. The skin provides protection from harmful ultraviolet radiation and temperature extremes. In addition, the skin also waterproofs the human body, provides a barrier protection from infection, excretes wastes and aids in the regulation of body temperature. The skin acts as the body's initial defense against foreign bodies such as bacteria, viruses and other potentially harmful microbes. Exposing skin to extreme temperatures causes burns and damage to the skin, and compromises the integrity and functionality of skin, specifically in the protection and maintenance of homeostasis in the body. Damage to the skin may result in the body being susceptible to harmful foreign bodies. Thus, compositions, methods, methods for dispensing and kits are needed for protection of the skin prior to exposure to extreme temperatures.

### SUMMARY OF THE INVENTION

The present disclosure teaches compositions used for the protection of human and/or animal skin, human contact materials or living organic tissue with temporary protection for exposures to temperatures from −140° F. to 3500° F. or higher. The composition can be placed on adjacent surfaces to prevent burns of surrounding materials as a temporary solution to cold or hot.

In one embodiment, the disclosure teaches a method for protecting skin from external thermal flux comprising applying a temperature-protective composition to the skin. The disclosure teaches a method for protecting skin from external thermal flux comprising applying the temperature-protective composition to the skin. The disclosure teaches a method for protecting a subject from extreme temperatures comprising placing a dispensing container in proximity to conditions of extreme temperatures, having the dispensing container contain the composition of claim 1, applying the composition to the skin of a subject prior to exposure of the skin to extreme temperatures, and then having the subject exposed to extreme temperature. The method may have the dispensing container selected from the group consisting of wall-mounted dispensers, aerosol or hand spray containers, screw-top refillable squeeze bottles, screw-top, refillable hand-held pump containers, individual one time use “tear and use” packages, tooth paste tube like packaging, multi-use clear containers, flip-top squeeze tubes, screw-top tub, material wipes stored in a sealed or resealable package, resealable topped cans or jars which can accommodate brush on applications, and wipe applicators. The application of the composition to the skin is mostly uniform and has a thickness of about 1 to 5 mm (0.039 to 0.197 in).

The instant disclosure relates to compositions that are unique in their ability to protect the skin from extreme temperatures. The compositions are useful in environments such as firefighting, outdoor, sporting, racing, automotive, showmen, stuntmen, commercial, industrial, and military environments, in addition to applications wherein exposure to extreme heat or cold is likely or possible. For instance, the compositions are useful in methods for protecting skin from intense external thermal flux (e.g., fires and explosives). The compositions are also unique in their dispensability and availability to persons operating in such environments.

Embodiments of the present invention use silica due to a characteristically low thermal conductivity. Aerogel is a synthetic porous ultralight material derived from a gel, in which the liquid component for the gel has been replaced with a gas. The result is a solid with extremely low density and extremely low thermal conductivity, resulting in an excellent thermal insulator. Certain variants of aerogels have a coefficient of thermal conductivity of approximately 0.024 W/mK at 20° C. In contrast, other well-known and commonly used thermal insulating materials have the following thermal conductivity values at a temperature of 20° Celsius: Extruded PolyStyrene: 0.033-0.046 W/mK; Air: 0.026 W/mK; Fiberglass: 0.045 W/mK. Although each of these thermal insulative materials provide thermal insulative benefits in certain applications, the use of such materials to protect the skin of a subject are unknown due to the physical properties of these materials preventing flexible use and application.

The present invention is directed toward overcoming one or more of the problems discussed above by providing a temperature-protective composition which can be applied to a subject's skin.

The users and applications where temperature-protective compositions are helpful to protect them against injures from fire, extreme heat and cold temperatures include, but are not limited to fireman, smoke jumpers, welders, plumbers, smelters, stuntmen, music performers and showman, power plant, refinery, chemical plant workers, policeman, race car, boat, motorcycle, airplane racers and crew, winter mountain and cold water rescue teams, commercial fisherman and skin divers, utility workers, city/town employees, transportation workers, oil field workers, snow removal personal, ski resort employees, construction workers, motorcycle riders, winter sport athletes, their fans and outdoor winter activities=football players, winter hikers, skiers, snow boarders, runners, cyclists, walkers, skin divers, surfers, pond hockey players, sledders, ice fisherman, snowmobilers, outdoor winter enthusiast, motorcycle drivers/rides, polar event swimmers, cold water kayaking, hunters and survivalists.

Although the present disclosure discusses the use of temperature-protective compositions herein for the purposes of protecting skin, temperature-protective compositions discussed herein can be used to protect material from fire damage, wood, natural and synthetic textiles, paper, plastic, paint, plants, metal, ceramic, glass, synthetic and natural leathers while in keeping with the spirit and scope of the present invention.

These and other advantages will be apparent from the disclosure of the inventions contained herein. The above-described embodiments, objectives, and configurations are neither complete nor exhaustive. As will be appreciated, other embodiments of the invention are possible using, alone or in combination, one or more of the features set forth above or described in detail below. Further, this Summary is neither intended nor should it be construed as being representative of the full extent and scope of the present invention. The present invention is set forth in various levels of detail in this Summary, as well as in the attached drawings and the detailed description below, and no limitation as to the scope of the present invention is intended to either the inclusion or non-inclusion of elements, components, etc. in this Summary. Additional aspects of the present invention will become more readily apparent from the detailed description, particularly when taken together with the drawings, and the claims provided herein.

### BRIEF DESCRIPTION OF THE DRAWINGS

FIG. 1A—Certain embodiments of a temperature-protective composition applied to the skin of a subject.

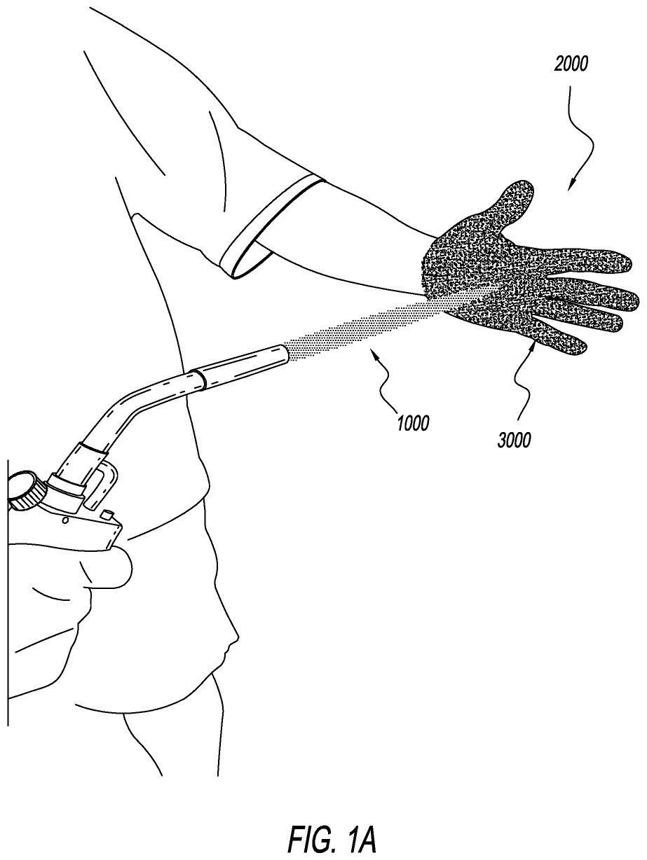

FIG. 1B—Certain embodiments of a temperature-protective composition applied to the skin of a subject.

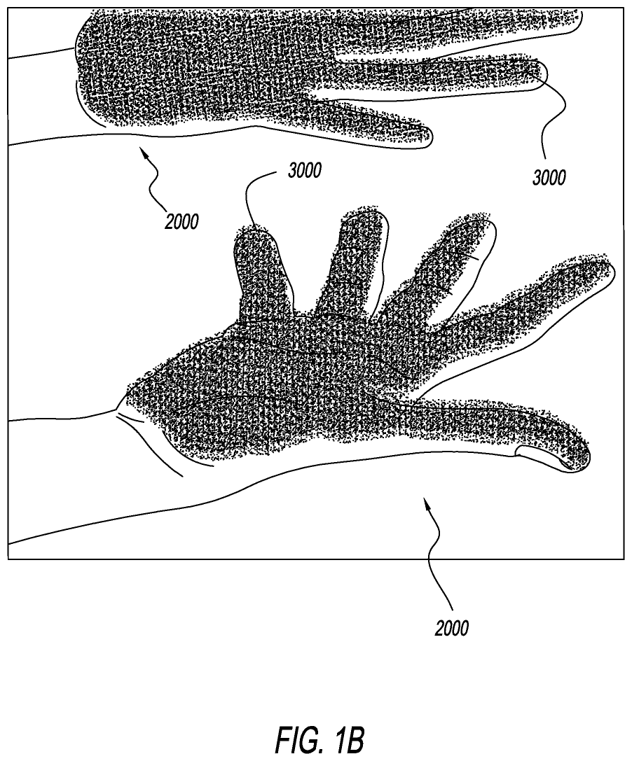

### DETAILED DESCRIPTION OF VARIOUS EMBODIMENTS

Certain embodiments comprise a temperature-protective composition comprising, by weight: 35-45% silica; 15-20% polyvinyl alcohol; 30-40% water; and 10% clay; wherein the weight percentages are based on the total weight of the temperature-protective composition. The silica can be a silica aerogel. Certain embodiments of a temperature-protective composition comprise surfactants.

In certain embodiments, a temperature-protective composition comprises by weight: 40% silica aerogel; 20% polyvinyl alcohol; and 40% water; wherein the percentages are based on the total weight of the temperature-protective composition.

While various aspects and features of certain embodiments have been summarized above, the following detailed description illustrates a few embodiments in further detail to enable one of skill in the art to practice such embodiments. The described examples are provided for illustrative purposes and are not intended to limit the scope of the invention.

Certain embodiments surround a temperature-protective composition comprising, by weight: (a) silica; (b) a binder; (c) optionally, a viscosity modifier; (d) water, and (e) optionally, other additives including surfactants. For example, certain embodiments comprising a temperature-protective composition include by weight: (a) 35-45% silica; (b) 15-25% polyvinyl alcohol; (c) optionally, 5-15% clay; (d) 20-40% water and (d) optionally, 0-10% other additives, wherein the weight percentages are based on the total weight of the temperature-protective composition. It will be appreciated that temperature-protective compositions can provide protection from both extreme heat and extreme cold environments.

In certain embodiments, temperature-protective compositions comprise, by weight: (a) 35% silica; (b) 15% polyvinyl alcohol; (c) 10% clay; and (d) 40% water, wherein the weight percentages are based on the total weight of the temperature-protective composition.

In certain embodiments, a temperature-protective composition comprises: (a) 40% silica; (b) 20% polyvinyl alcohol; and (c) 40% water, wherein the weight percentages are based on the total weight of the temperature-protective composition. It will be appreciated that temperature-protective compositions can provide protection from both extreme heat and extreme cold environments.

Certain embodiments of a temperature-protective composition comprise silica. The following are the (by weight) percentage of silica comprised by various embodiments: (a) 30-50%; (b) 30-45%; (c) 30-35%; (d) 35-50%; (e) 35-45%; or (f) 35-40%.

Various embodiments of a temperature-protective composition comprises a binder. The following are the (by weight) percentage of binder comprised by various embodiments: (a) 15-25%; (b) 15-%, or (c) 20-25%.

Various embodiments of a temperature-protective composition comprises a viscosity modifier. The following are the (by weight) percentage of viscosity modifier comprised by various embodiments: (a) 5-15%; (b) 5-10%; (c) 5-8%; (d) 10-15%; or (e) 12-15%.

The silica as referenced herein includes but is not limited to: silicon dioxide, quartz, tridymite, cristobalite, coesite, keatite, lechatelierite, silica sand, sandstone, ceramics, silicon carbide, ferrosilicon, and silicones, silicone oils, refractory material as in gemstones and silica gels. The insulating component: (silica, polymer, carbon based of particle size 500 nm to 2 mm), a (e.g., a solid and hollow glass microspheres, microspheres, ceramic microsphere, microsphere made form fly ash, a mica, a wollastonite, a silica, a barium sulfate, a metal powder (e.g., a metal oxide), a carbon black, fumed silica, fumed alumina, a talc, a fiber (e.g., a cellulose fiber, a cotton fiber, a wood flour, a carbon fiber, a glass fiber), a microsphere may be between about 4 μm to about 5000 μm in diameter; though a hollow microsphere are generally up to about 200 μm in diameter. This list is not intended to be limiting in defining the silica component.

The binder is described by but is not limited to: polyvinyl alcohol solution in water: including Poly(vinyl alcohol) (PVOH, PVA, or PVAl) as a water-soluble synthetic polymer, with the formula [CH2CH(OH)], poly vinyl alcohol, polyvinyl acetate, copolymers with any acrylic acid esters, polyurethanes, polyurea's, silicone's, polyesters, polyethylene oxides, poly propylene oxides and their copolymers, polymer and copolymer of fluorinated monomers, emulsion or dispersion of polymers in water and solvents, and mixtures of above polymers in different ratios.

Embodiments including a viscosity modifier have a viscosity modifier comprising clay. Alternative embodiments comprise a viscosity modifier comprising minerals, hydrous aluminum phyllosilicates, variable amounts of iron, magnesium, alkali metals, alkaline earths, and other cations (“kaolin”), bentonite, montmorillonite, attapulgite clay, a sand/quartz powder, a calcium carbonate (e.g., limestone), calcium sulfate (gypsum) a whiting, or a combination thereof—while in keeping with the spirit and scope of the present embodiment.

Other additives is described by but is not limited to: surfactants having a chemical structure having a polar head group and a hydrophobic tail, anionic surfactants, cationic surfactants, non-ionic surfactants, phospholipids, and zwitterionic surfactants.

The temperature-protective compositions described herein may include one or more silica, binders, viscosity modifiers, other additives or combinations thereof.

Certain embodiments of a temperature-protective composition comprise a gel form while others comprise: an emulsion, a cream, a foam, an aerogel, a lotion, a paste, oil and/or ointment, or combinations thereof. Embodiments of a temperature-protective composition as described herein are applied to the skin topically.

Application of the temperature-protective composition is mostly uniform on the area of skin that is in need of protection. Certain embodiments require an application of the temperature-protective composition in thicknesses of between 1-5 mm (0.039-0.197 in). Certain embodiments require an application of the temperature-protective composition in thicknesses of between 1-4 mm (0.039-0.157 in). Certain embodiments require an application of the temperature-protective composition in thicknesses of between 1-3 mm (0.039-0.118 in). Certain embodiments require an application of the temperature-protective composition in thicknesses of between 1-2 mm (0.039-0.079 in). Certain embodiments require an application of the temperature-protective composition in thicknesses of between 0.1-6 mm (0.0039-0.236 in). It will be appreciated that the applied thickness of the coating varies dependent upon the area of skin to be exposed to extreme temperatures.

Certain embodiments comprise a dispenser for holding and applying a temperature-protective material. Such dispensers include, but are not limited to: wall-mounted dispensers, aerosol or hand-spray containers, screw-top refillable squeeze bottles, screw-top containers, hand-held pump containers, refillable containers, individual one time use “tear and use” packages, toothpaste tube-like containers, multi-use clear containers, flip-top squeeze tubes, screw-top tub, material wipes stored in a sealed or resealable package, resealable topped cans or jars which can accommodate brush on application, or packaged material wipes. Temperature-protective material of certain embodiments can be dispensed from a “soap like dispenser”, such that the dispenser is attached to the wall in a dressing room or workstations. The temperature-protective material of certain embodiments can be dispensed from aerosol or hand spray container for quickly spraying onto skin. The temperature-protective material of certain embodiments can be dispensed from a screw-top bottle. Certain embodiments comprise a dispenser which is clear such that the amount of material in the dispensers, or other features of the material, is determined by visual inspection without opening the dispenser. Other features of the material such as by color coding or visual appearance of the texture of the material can be inspected through clear dispensers.

### EXAMPLES

The following examples are provided for illustrative purposes only and are not intended to limit the scope of the invention.

### Example 1

Formulation 1: (Percentages are by Weight)

| | |
|---|---|
| Silica | 30-45% |
| Binder | 10-20% |
| Water | 30-40% |
| Viscosity Modifier | 5-10% |
| Other Additives | 0-25% |

Formulation 2: (Percentages are by Weight)

| | |
|---|---|
| Silica | 35-45% |
| Polyvinyl alcohol | 15% |
| Water | 30-40% |
| Clay | 10% |

Formulation 3: (Percentages are by Weight)

| | |
|---|---|
| Silica Aerogel | 40% |
| Polyvinyl alcohol | 20% |
| Water | 40% |

Certain embodiments of a temperature-protective composition, such as formulations 1-3 for instance, were tested for their ability to protect skin from extreme heat wherein a flame 1000 was brought in near proximity to a subject's hand 2000, wherein the temperature-protective composition 3000 was applied to the subject's hand 2000. The temperature-protective composition 3000 was applied the hand 2000 to provide coverage of the hand 2000 with a substantially uniform thickness coating. Following application of formulation 2 to a subject's hand 2000, the hand 2000 was exposed to extreme heat from a flame 1000 having a temperature between 815-1927° C. (1500-3500° F.). The skin of the hand 2000 did not have any damage during or after exposure to the extreme heat.

### Example 2: Protection for Power Plant, Refinery, Chemical Plant Workers

Power plant, refinery, and chemical plant workers work around steam pipes and other equipment that expose them to temperatures in excess of 538° C. (1000° F.). Worker injuries are caused by accidental contact to super-heated pipes and equipment, and steam purging from pipes. A temperature-protective composition in a “soap like dispenser” attached to the wall of a dressing room or on wall near a workstation provides an easy way to apply temperature-protective composition to their skin for protection from the extreme heat to which they are exposed. Other methods of applying temperature-protective composition could include: temperature-protective composition in aerosol or hand spray container for quickly spraying on to skin, temperature-protective composition in screw-top refillable squeeze bottle for hand applying material, temperature-protective composition in screw-top, in refillable hand held pump container, temperature-protective composition in individual one time use “tear and use” packages for applied use, temperature-protective composition in individual—toothpaste tube-like packaging—clear packaging showing the amount of product remaining, temperature-protective composition in flip-top squeeze tube for hand applying—clear tube shows amount of or type of product remaining in package, temperature-protective composition in screw-top tub—for hand applying, protective fabric material wipes stored in resealable packaging.

### Example 3: Protection for Military Personnel Skin

Military personnel are exposed to many different extreme hot and cold temperatures, whether in battle or working with equipment associated with jet engines, ship engines, power plants, weapons in addition to exposure to winter conditions. The use of a temperature-protective gel can help prevent burns, frostbite and hypothermia. Temperature-protective composition in a “soap like dispenser” attached to the wall of their dressing room or on a wall near a workstation or in a vehicle, would provide an easy way to apply temperature-protective composition onto their skin for protection from the extreme temperatures to which they are exposed. other methods of applying temperature-protective composition could include: temperature-protective composition in aerosol or hand spray container for quickly spraying on to skin, temperature-protective composition in screw-top refillable squeeze bottle for hand applying material, temperature-protective composition in screw-top refillable hand held pump container, temperature-protective composition in individual one time use “tear and use” packages for hand applied use, temperature-protective composition in individual toothpaste tube-like packaging—clear packaging shows the type or amount of product remaining, temperature-protective composition in flip-top squeeze tube for hand applying—clear tube shows amount of product remaining in package, temperature-protective composition in screw-top tub for hand applying, temperature-protective fabric material wipes stored in re-sealable packaging.

### Example 4: Protection for Sportsmen from Extreme Cold

Protection from extreme cold (frostbite—hypothermia) for winter sport athletes, their fans and outdoor winter activities such as football players, winter hikers, skiers, snow boarders, runners, cyclists, walkers, skin divers, surfers, pond hockey players, shoveling, sledders, ice fisherman, snowmobilers, outdoor winter enthusiast, motorcycle drivers/rides, polar event swimmers, cold water kayaking, hunters, pond hockey players. Methods of applying temperature-protective composition could include: wall-mounted soap type dispenser filled with temperature-protective composition, temperature-protective composition in aerosol or hand spray container for quickly spraying on to skin, temperature-protective composition in screw-top refillable squeeze bottle for hand applying material, temperature-protective composition in screw-top refillable hand-held pump container, temperature-protective composition in individual one time use “tear and use” packages for hand-applied use, temperature-protective composition in individual—toothpaste tube-like packaging, clear packaging showing type or amount of product remaining, temperature-protective composition in flip-top squeeze tube for hand applying, clear tube showing type or amount of product remaining in package, temperature-protective composition in screw-top tub for hand applying, temperature-protective fabric material wipes stored in resealable packaging.

### Example 5: Protection for Auto, Motorcycle, Boat, Airplane Racers and their Crews from Extreme Heat and Flames

Racers and crews are exposed to extreme heat from engine and fuel fires. A temperature-protective composition in a “soap like dispenser” attached to the wall of their dressing room or crew bus would provide an easy way to get temperature-protective composition onto their skin help protect them from these dangers. Other methods of applying temperature-protective composition could include: temperature-protective composition in aerosol or hand spray container for quickly spraying on to skin, temperature-protective composition in screw off top re-fillable squeeze bottle for hand applying material, temperature-protective composition in screw off top—refillable hand held pump container, temperature-protective composition in individual one time use “tear and use” packages for hand applied use, temperature-protective composition in individual—tooth paste like packaging—clear packaging shows amount of product remaining, temperature-protective composition in flip top squeeze tube for hand applying—clear tube shows amount of product remaining in package, temperature-protective composition in screw off top tub—for hand applying, temperature-protective Fabric Material wipes stored in re-sealable packaging.

### Example 6: Protection from Extreme Heat from Motorcycle and Automobile Exhaust Equipment

Leg burns from Motorcycle exhaust pipes and automobile side exhaust systems are very common—especially in the summer. Applying temperature-protective compositions to the skin area in close proximity to these exhaust systems would help prevent burns from accidental contact. Methods of applying temperature-protective composition include: temperature-protective composition in aerosol or hand spray container for quickly spraying on to skin, temperature-protective composition in screw off top re-fillable squeeze bottle for hand applying material, temperature-protective composition in screw off top—refillable hand held pump container, temperature-protective composition in individual one time use “tear and use” packages for hand applied use, temperature-protective composition in individual—tooth paste like packaging—clear packaging shows amount of product remaining, temperature-protective composition in flip top squeeze tube for hand applying—clear tube shows amount of product remaining in package, temperature-protective composition in screw off top tub—for hand applying, temperature-protective fabric material wipes stored in re-sealable packaging.

### Example 7: Protection from Extreme Heat for Stuntmen, Music Performers and Showmen

Stuntmen, music performers and showmen often use fire to make their performances more exciting. Temperature-protective composition in a “soap like dispenser” attached to the wall of their dressing room or tour bus would provide an easy way to get temperature-protective composition onto their skin help protect them from burns. Other methods of applying temperature-protective composition could include: temperature-protective composition in aerosol or hand spray container for quickly spraying on to skin, temperature-protective composition in screw off top re-fillable squeeze bottle for hand applying material, temperature-protective composition in screw off top—refillable hand held pump container, temperature-protective composition in individual one time use “tear and use” packages for hand applied use, temperature-protective composition in individual—tooth paste like packaging—clear packaging shows amount of product remaining, temperature-protective composition in flip top squeeze tube for hand applying—clear tube shows amount of product remaining in package, Temperature-protective composition in screw off top tub—for hand applying, Temperature-protective Fabric Material wipes stored in re-sealable packaging.

### Example 8: Temperature-Protective Composition Placed in Exit Stairs Emergency Cases—Protection from Fire and Extreme Heat

Building codes require emergency equipment to be placed at egress points for occupants of a building seeking to escape from a fire and for emergency personnel for emergency use. Equipment such as fire hoses and axes are located within stairwells and near emergency exits. A temperature-protective composition within a nonflammable compressed-air spray cans, or other containers of temperature-protective material, placed in the emergency case for rapid application prior to entering an area of fire-danger or extreme heat offers added protection for getting through and away from the danger.

### Example 9: Home and Auto Personal-Protection from Fire and Extreme Heat

Temperature-protective compositions in various containers and applicators can be place in automobile glove-boxes, home/auto emergency kits, hiking/camping kits and in-home kitchen draws to help prevent injuries from fire and extreme heat. Dispensing means include: a temperature-protective composition in aerosol or hand spray container for quickly spraying on to skin, temperature-protective composition in screw-top refillable squeeze bottle for hand applying material, temperature-protective composition in screw-top refillable hand held pump container, temperature-protective composition in individual one time use “tear and use” packages for hand applied use, temperature-protective composition in individual—tooth paste like packaging—clear packaging shows amount of product remaining, temperature-protective composition in flip top squeeze tube for hand applying—clear tube shows amount of product remaining in package, temperature-protective composition in screw off top tub—for hand applying, temperature-protective Fabric Material wipes stored in re-sealable packaging.

The description of the various embodiments has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiments described and shown in the figures were chosen and described in order to explain the principles of the invention, the practical application, and to enable others of ordinary skill in the art to understand the invention for various embodiments with various modifications as are suited to the particular use contemplated. All references cited herein are incorporated in their entirety by reference.

In the following description, for the purposes of explanation, numerous specific details are set forth in order to provide a thorough understanding of the described embodiments. It will be apparent to one skilled in the art, however, that other embodiments of the present invention may be practiced without some of these specific details. Several embodiments are described and claimed herein, and while various features are ascribed to different embodiments, it should be appreciated that the features described with respect to one embodiment may be incorporated with other embodiments as well. By the same token, however, no single feature or features of any described or claimed embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

The term “aerogel” refers to a synthetic, porous, ultralight material derived from a gel, in which the liquid component of the gel is replaced with a gas.

The term “silica” refers to silicon dioxide, compounds, synthetic products, polymorphs, and minerals.

The term “silica aerogel” refers to a silica-based aerogel derived from silica gel.

The term “skin” is used to describe human and or animal skin.

The term “topical” as used herein refers to direct application to the skin, in terms of dermatological compositions being applied to the skin. Examples of topical applications include gels, aerogels, semisolids, solutions to spray, liquids to be applied by an applicator, lotions, and foams.

It will be appreciated that ranges of various embodiments are inclusive and combinable. For examples, any value or point described herein that falls within a range described herein can serve as a minimum or maximum value to derive a sub-range, etc.

While various embodiments of the present invention have been described in detail, it is apparent that modifications and alterations of those embodiments will occur to those skilled in the art. However, it is to be expressly understood that such modifications and alterations are within the scope and spirit of the present invention. Further, the inventions described herein are capable of other embodiments and of being practiced or of being carried out in various ways. In addition, it is to be understood that the phraseology and terminology used herein is for the purposes of description and should not be regarded as limiting. The use of “including,” “comprising,” or “adding” and variations thereof herein are meant to encompass the items listed thereafter and equivalents thereof, as well as, additional items.

What is claimed is:

1. A temperature-protective composition comprising:
silica;
polyvinyl alcohol; and
water.

2. The temperature-protective composition of claim 1, wherein the silica comprises 30-50% of the temperature-protective composition by weight.

3. The temperature-protective composition of claim 2, wherein:
the polyvinyl alcohol comprises 20% by weight; and
the water comprises 40% by weight.

4. The temperature-protective composition of claim 1, wherein the silica is silica aerogel.

5. The temperature-protective composition of claim 1, wherein:
the silica comprises 35-45% by weight; and
the clay comprises 10% by weight.

6. The temperature-protective composition of claim 1, further comprising clay.

7. The temperature-protective composition of claim 6, wherein:
the silica comprises 35-45% by weight; and
the clay comprises 10% by weight.

8. The temperature-protective composition of claim 7, wherein:
the polyvinyl alcohol comprises 15-20% by weight; and
the water comprises 30-40% by weight.

9. A method for protecting skin from extreme temperatures comprising applying the temperature-protective composition of claim 8 to a skin of a subject.

10. The method of claim 9 wherein the application of the temperature protective composition is applied in a thickness between 1-5 mm ((0.039 to 0.197 in).
